(19) United States
(12) Patent Application Publication
(10) Pub. No.: US 2025/0041049 A1
(43) Pub. Date: Feb. 6, 2025

(54) THIN LARGE-DIAMETER OPTICS FOLDABLE INTRAOCULAR LENS

(71) Applicants: University of Rochester, Rochester, NY (US); Oregon Health & Science University, Portland, OR (US)

(72) Inventors: David Huang, Portland, OR (US); Scott MacRae, Rochester, NY (US)

(21) Appl. No.: 18/715,340

(22) PCT Filed: Dec. 5, 2022
(86) PCT No.: PCT/US2022/051830

§ 371 Date: May 31, 2024

Related U.S. Application Data
(60) Provisional application No. 63/286,356, filed on Dec. 6, 2021.

(51) Int. Cl. A61F 2/16

(52) U.S. Cl. CPC A61F 2/1656; A61F 2002/169053; A61F 2240/001

(57) ABSTRACT

Foldable intraocular lens (IOL) having a total dioptric power and comprising an optic portion (12) having main anterior and posterior optical surfaces, and incorporating an internal optical feature (13) positioned between the main anterior and posterior optical surfaces are described. The optic portion has a diameter of greater than 6.0 mm and a maximum central cross-sectional area of less than 2 mm² along the diameter, and the internal optical feature positively contributes to the total dioptric power of the intraocular lens. A method of forming the IOL may include: providing a foldable IOL having an optic portion comprising an optical, polymeric lens material and having an anterior surface and posterior surface and an optical axis intersecting the surfaces; and forming at least one laser-modified layer disposed between the anterior surface and the posterior surface with light pulses from a laser by scanning the light pulses along regions of the optical, polymeric material to cause changes in the refractive index of the polymeric lens material; wherein the laser-modified layer forms the internal optical feature that positively contributes to the total dioptric power of the IOL. A method of inserting the IOL into an eye may include folding the IOL, making an incision of less than 3.0 mm length, and inserting the folded IOL through the incision.

### FIELD

The disclosure is directed towards a medical device in the form of an intraocular lens designed for small-incision surgery, which also reduces the risk of dysphotopsia symptoms after cataract surgery.

### BACKGROUND

Intraocular lens (IOL) implantation is a routine part of cataract surgery. The IOL is typically placed within the lens capsular bag and replaces the function of the natural crystalline lens to focus an image onto the retina in the back of the eye. Modern cataract surgery is performed through a small limbal incision with width preferably between 2 to 3 mm, or even smaller. Such a small incision is important for reduced induction of astigmatism, self-sealing without sutures, reduced discomfort, and faster healing and visual recovery. In order to be inserted through such a small incision, the IOL must be sufficiently thin and flexible to be foldable with a cross-sectional area sufficiently small to pass through an injector device and the limbal incision, and is typically of relatively small diameter to enable such sufficiently small cross-sectional area at desired optical lens powers. The typical IOL diameter presently employed, e.g., is 6.0 mm. Unfortunately, the small diameter carries with it a high risk of dysphotopsia symptoms after surgery. Dysphotopsia refers to light or shadows seen in the periphery of vision under certain lighting conditions. Although some patients learn to ignore this phenomenon, it can be persistent and disturbing to others. The cause is unfocused light that is projected through a gap between the outer edge of the IOL and inner edge of the iris.

A larger IOL diameter would help reduce or eliminate the gap between the IOL and iris edges, and reduce the risk of dysphotopsia. Although IOLs with optics diameter greater than 6.0 mm, e.g., of 6.5 mm and 7.0 mm, are available, they are rarely used because of the need to enlarge limbal incisions to greater than 3.0 mm for insertion of such currently available larger IOLs, as it is difficult to increase the diameter of a foldable conventional IOL for desired relatively high optical power lenses and still have it fit through a small incision, as such larger diameter IOL typically require increased center thickness and resulting cross-sectional area so as to maintain a desired dioptric power obtained by refraction associated with the base surface curvatures of the optics. Making such a large diameter IOL thin enough to be foldable and insertable through such a small incision opening instead limits the dioptric power of a conventional refractive IOL obtained by refraction associated with the base surface curvatures of the optics to below the commonly needed values.

Nordan et al. U.S. Pat. No. 6,152,958 and Silberman et al. U.S. Pat. No. 5,178,636 disclose the use of a relatively thin IOL with increased power or multifocality obtained using a Fresnel lens design. However, such a Fresnel lens with surface ridges may cause scattering and a tendency to trap material in the recesses. Forward scattering, e.g., can fog image quality while side scattering can produce glare. These disclosures further only suggests IOL diameters up to 6 mm.

Thus it would be desirable to provide relatively large diameter, relatively thin IOLs enabling relatively high dioptric powers, without requiring formation of surface ridges.

### SUMMARY

According to aspects illustrated herein, there is provided a foldable intraocular lens (IOL) having a total dioptric power and comprising an optic portion having main anterior and posterior optical surfaces, and incorporating an internal optical feature positioned between the main anterior and posterior optical surfaces, wherein the optic portion has a diameter of greater than 6.0 mm and a maximum central cross-sectional area of less than 2 mm² along the diameter, and wherein the internal optical feature positively contributes to the total dioptric power of the intraocular lens.

According to other aspects, a method of forming an intraocular lens having a total dioptric power is described comprising:
providing a foldable intraocular lens having an optic portion comprising an optical, polymeric lens material and having an anterior surface and posterior surface and an optical axis intersecting the surfaces; and
forming at least one laser-modified layer disposed between the anterior surface and the posterior surface with light pulses from a laser by scanning the light pulses along regions of the optical, polymeric material to cause changes in the refractive index of the polymeric lens material;
wherein the optic portion has a diameter of greater than 6.0 mm and a maximum central cross-sectional area of less than 2 mm² along the diameter, and wherein the laser-modified layer forms an internal optical feature that positively contributes to the total dioptric power of the intraocular lens.

According to other aspects, there is disclosed herein a method of inserting an intraocular lens into an eye comprising making an incision of less than 3.0 mm length, and inserting a folded intraocular lens through the incision, wherein the intraocular lens has a total dioptric power and comprises an optic portion having main anterior and posterior optical surfaces, a diameter of greater than 6.0 mm and a maximum central cross-sectional area of less than 2 mm² along the diameter, and an internal optical feature positioned between the main anterior and posterior optical surfaces, wherein the internal optical feature positively contributes to the total dioptric power of the intraocular lens.

### BRIEF DESCRIPTION OF THE DRAWINGS

FIG. 1. En face diagram of one embodiment of an IOL of the present disclosure showing gradient index of refraction.

FIG. 2. Cross-sectional diagram of one embodiment of an IOL of the present disclosure showing gradient index of refraction.

FIG. 3. En face diagram of one embodiment of an IOL of the present disclosure showing gradient index of refraction in concentric rings analogous to a Fresnel lens.

FIG. 4. Cross-sectional diagram of one embodiment of an IOL of the present disclosure showing gradient index of refraction in concentric rings analogous to a Fresnel lens.

FIG. 5. Cross-sectional refractive index profile showing variation of index of refraction in concentric rings analogous to surface height variation in a Fresnel lens.

FIGS. 6a and 6b (Prior Art) provide an illustration of a modulo-2pi phase wrapped Fresnel lens (FIG. 6b), for a corresponding conventional continuous refractive lens (FIG. 1a).

FIG. 7 is a schematic of a laser system which may be used for writing an internal optical feature in an intraocular lens in accordance with an embodiment.

### DETAILED DESCRIPTION

The disclosure is directed towards intraocular lenses (IOLs) incorporating an internal optical feature positioned between main anterior and posterior optical surfaces of the IOL, wherein the internal optical feature is designed to positively contribute to the total dioptric power of the IOL. By providing such an internal optical feature, the IOL optics diameter may be increased to greater than conventional 6 mm optics diameter, while maintaining the IOL central cross-sectional area to less than 2 mm² along the lens diameter, while still providing a desired total dioptric power, such that the thin, large diameter IOL with desired total dioptric power may still be inserted through a limbal incision of 3.0 mm or less (more preferably through a limbal incision of 2.6 mm or less, or of 2.2 mm or less, or of 2.0 mm or less, or of 1.5 mm or less, with exemplary incisions in some embodiments down to, e.g., 1.0 mm).

The modern foldable IOL is typically rolled and injected through a round injector tube. The injector tube is fit through an incision of length l_{incision} near the corneal limbus. The typical incision length for modern cataract surgery is between 2 to 3 mm. A smaller incision length has the advantage of reducing the chance of wound leakage without suturing and reducing the magnitude of incision-induced astigmatism. Since the injector tube outside circumference must fit inside the incision, the injector lumen internal cross-sectional area σ₁, in mm², is given by the formula below:

$${σ}_{I} = {π ( \frac{{l}_{incision}}{π} - w )}^{2}$$

where w is the injector wall thickness in mm.

Using the above formula, Table I below presents injector lumen calculated internal cross-sectional areas for incision lengths of from 2 to 3 mm and a typical injector wall thickness of 5% of the injector tube's outer diameter.

TABLE I

| | Injector wall | Injector |
|---|---|---|
| Incision | thickness | Cross section |
| (mm) | (mm) | (mm²) |
| 3 | 0.15 | 2.036892 |
| 2.9 | 0.145 | 1.903362 |
| 2.8 | 0.14 | 1.774359 |
| 2.7 | 0.135 | 1.649883 |
| 2.6 | 0.13 | 1.529932 |
| 2.5 | 0.125 | 1.414508 |
| 2.4 | 0.12 | 1.303611 |
| 2.3 | 0.115 | 1.19724 |
| 2.2 | 0.11 | 1.095395 |
| 2.1 | 0.105 | 0.998077 |
| 2 | 0.1 | 0.905285 |

A preferred goal for the present invention is for the IOL to fit through an incision length of 2.6 mm or less, which corresponds to a calculated internal lumen cross-sectional area of 1.53 mm² or less. The IOL of the presentation invention accordingly should preferably have a sagittal cross-sectional area smaller than that. In further embodiments, it may be further preferable for the IOL to fit through even smaller incision lengths (e.g., of 2.2 mm or less, or 2.0 mm or less, or 1.5 mm or less), with the IOL having a sagittal cross sectional area smaller than the corresponding calculated internal lumen cross-sectional area for such an incision (e.g., IOL maximum cross-sectional area of less than or equal to about 1.1 mm² for an incision of 2.2 mm or less, or IOL maximum cross-sectional area of less than or equal to about 0.91 mm² for an incision of 2.0 mm or less).

To calculate the sagittal cross-sectional area of an IOL, the following equations are used.

The central thickness s (for sagittal height) of the intraocular lens (IOL) in mm is given by

$$s = {t}_{e a g e} + {( \frac{d}{2} )}^{2} \frac{P}{2000 ( n - 1 )}$$

where t_{edge} is the IOL edge thickness in mm, d is the IOL diameter in mm, P is the lens power in air in diopters, and n is the IOL refractive index. This calculation assumes that the lens profile is near spherical and the edge is a straight truncation. This closely approximate actual typical IOL designs for the purpose of cross-sectional area calculations.

The IOL cross-section, σ, in mm² is given by

$$σ = d ( \frac{1}{3} {t}_{edge} + \frac{2}{3} s )$$

In one preferred embodiment of the present invention, the power of the IOL is provided in the conventional way based on curvature of the main anterior and posterior optical surfaces of the IOL up to a maximum amount Pₓ, at which the cross-sectional area of the IOL is within a desired limit so as to be insertable through a desired incision length, and beyond which additional power is provided by an internal optical feature positioned between the main anterior and posterior optical surfaces of the IOL, such as an internal refractive index gradient. By limiting Pₓ, and providing additional power by employing an internal optical feature, a larger diameter than the customary 6 mm can be used in higher powered IOLs without exceeding the target limit on cross-sectional area of less than 2 mm², and preferably less than 1.53 mm². In one example, for an IOL having d=7 mm, Pₓ=17 diopters, n=1.47, and t_{edge}=0.02 mm, according to the formula, the cross-sectional area is 1.17 mm², well within the preferred target limit. In another example, for an IOL having d=8 mm, Pₓ=15 diopters, n=1.55, and t_{edge}=0.02 mm, according to the formula, the cross-sectional area is 1.32 mm², still below the preferred target limit.

In specific embodiments, the internal optical feature which positively contributes to the total dioptric power of the intraocular lens is designed so as to enable an IOL with total dioptric power from about 5 D to about 40 D in aqueous and vitreous humors of the human eye, an optics diameter of at least 6.1 mm, preferably at least 6.3 mm and more preferably at least 6.5 mm, and up to, e.g., 10 mm, more typically up to 9 mm and most typically up to 8 mm. Preferred exemplary optics diameter ranges may include, e.g., from 6.1-10 mm, more preferably 6.3 to 9.0 mm, and more preferably 6.5 to 8.0 mm, 6.5 to 7.5 mm, or 6.5 to 7.0 mm, while having a maximum IOL central cross-sectional area of less than 2 mm² along the IOL diameter, preferably less than or equal to 1.53 mm², less than or equal to 1.3 mm², or less than or equal to 1.0 mm², wherein the internal optical feature is designed to provide a significant percentage (e.g., in certain embodiments at least 20%, or at least 33%, or at least 50%) of the total dioptric power of the IOL. In certain embodiments, the internal optical feature may be designed to provide a majority of the total dioptric power of the IOL (e.g., at least 51% of the IOL total dioptric power). By enabling relatively higher dioptric powers while maintaining cross-sectional areas of such relatively large diameter lenses to below specified levels, the described IOLs employing an internal optical feature maintain the ability to be inserted through relatively small incisions of less than 3 mm, preferably less than or equal to 2.6 mm, and more preferably less than or equal to 2.2 mm, or less than or equal to 2.0 mm, or less than or equal to 1.5 mm.

In preferred embodiments, the IOL still includes curved optical surface while remaining under such maximum cross-sectional areas so as to provide a fraction of the IOL dioptric power. Conventionally available foldable IOL lens polymer materials are available with relatively high refractive indices (e.g., above about 1.4, more preferably above about 1.5) that allow for obtaining dioptric powers of, e.g., up to about +20 D in a foldable lens having a cross-sectional area of less than 2 mm² in a relatively large diameter (e.g., 7-8 mm) lens. In such embodiments, the present disclosure is directed towards use of an internal optical feature which provides added dioptric power, so as to enable even higher dioptric powers in a relatively large diameter IOL which is insertable through a relatively small incision. In certain embodiments, e.g., the main anterior and posterior optical surfaces may be convex surfaces providing a refractive optical power component of the total dioptric power of the intraocular lens of from about +5 D to about +20 D in aqueous and vitreous humors of the human eye, while the internal optical feature is designed to provide from about +1 D to about +20 D added dioptric power, more preferably from about +3 D to about +20 D added dioptric power, or from about +5 D to about +20 D dioptric power, such that the IOL provides a desired dioptric power of from about +6 D to about +40 D (or +8 D to +40 D, or +10 D to +40 D) dioptric power, while still enabling relatively large optics diameters of greater than 6 mm and cross-sectional areas of less than 2 mm², less than or equal to 1.53 mm², less than or equal to 1.3 mm², or less than or equal to 1.0 mm². In some embodiments, the IOL preferably has a total dioptric power of at least 10 D, at least 20 D, at least 25 D or at least 30 D (e.g., from 10 D to 40 D, from 20 D to 40 D, from 25 D to 40 D, or from 30 D to 40 D).

The internal optical feature may be in the form of diffractive optical elements, refractive optical elements, or a combination of diffractive and refractive optical elements.

In accordance with the present disclosure, however, the optical feature positively contributing to the total dioptric power of the IOL is specifically provided internally between the main anterior and posterior optical surfaces of the IOL, so as not to require introduction of surface ridges to the IOL.

In one embodiment, the internal optical feature may be in the form of diffractive optical features, such as concentric alternating rings of different refractive index formed similarly as in the pattern of a Fresnel Zone Plate, formed internally to the IOL surfaces. In one embodiment, e.g., for a monofocal intraocular lens, the rings of different refractive index may be designed to direct light within the visible spectrum completely or substantially completely to a single diffraction order and/or focus. It is to be noted that while diffractive optics may further be used for the purpose of generating multifocality, this is distinct from the use of diffractive elements to contribute to the total dioptric power of the IOL so as to enable thinner, larger diameter IOLs in accordance with the present disclosure. Multifocality, however, may be further achieved where desired in combination with use of internal optical features contributing to the total dioptric power of the IOL.

In another embodiment, the internal optical feature may be in the form of refractive optical elements, such as internal sections of the IOL forming a gradient index of refraction, or may be in the form of a combination of such refractive optical elements and further diffractive optical elements, e.g., to additionally provide multifocality (division of light between several focuses). As diffractive optics have some potentially undesirable limitations, however, in that they may diffract light into several modes which creates inefficiency, in a particular embodiment of the IOLs of the present disclosure the internal optical feature comprises only refractive elements, and is free of intentionally diffractive optical elements.

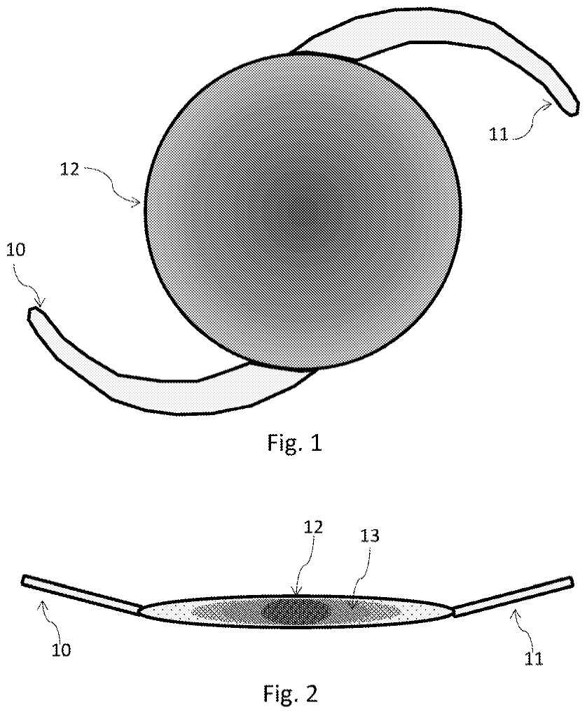

In a particular embodiment of the present disclose as shown in FIG. 1, e.g., an IOL comprises conventional haptics 10 and 11 but a novel design for the optics 12 in a relatively large diameter IOL that derives focusing power by both surface curvature and an internal optical feature comprising a gradient in refractive index formed in an internal layer extending between and generally parallel to the main surfaces of the IOL. As shown in FIG. 2, the surface curvature may be convex on either or both of the front and back surfaces, similar to a conventional IOL. However, the curvature can be maintained relatively small to allow the IOL optics to be made thin while the diameter is increased to greater than 6.0 mm. The gradient is radial, with the central portion having the highest index and the periphery having the lowest, so as to provide added dioptric power relative to that obtained only from the surface curvature of the IOL. By providing an internal optical feature formed with a gradient in the refractive index extending generally across the entire optic portion, a relatively higher dioptric power may be added to the lens without further increasing the thickness of the lens. Accordingly, a significant portion of the total dioptric focusing power of the IOL (e.g., at least 20%, more preferably from 20 to 80% of the IOL dioptric power) may be derived from the internal gradient index of refraction layer in such relatively thin IOL. Thus, the internal gradient index may be designed to significantly increase the dioptric power of the IOL in comparison to the dioptric power of an IOL having the same surface curvatures but without the internal gradient index of refraction.

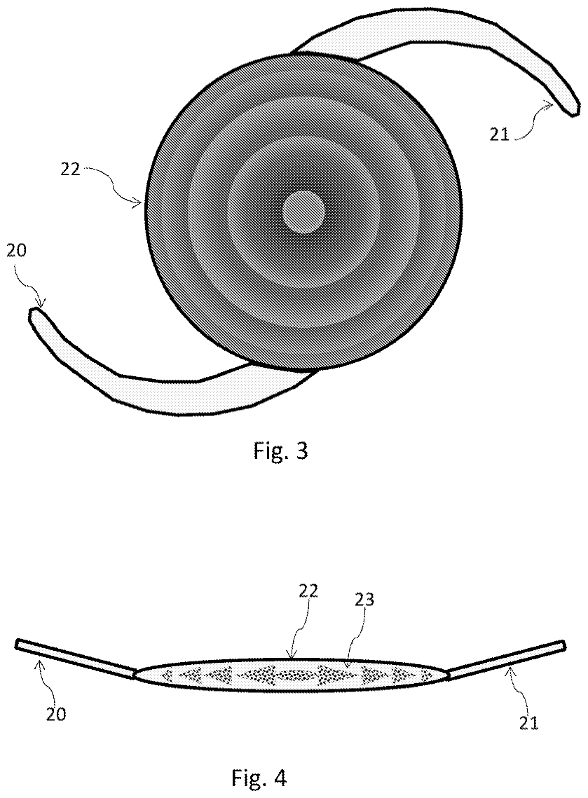

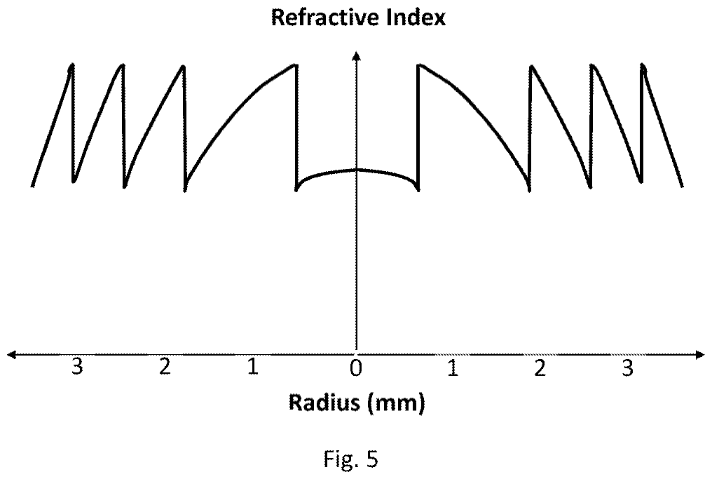

In another particular embodiment of the present disclosure as shown in FIG. 3, an IOL comprises conventional haptics 20 and 21 but a novel design for the optics 22 in a relatively large diameter IOL that derives focusing power by both surface curvature and an internal optical feature comprising concentric rings of internal gradient in refractive index formed in a layer extending between and generally parallel to the main surfaces of the IOL. As shown in FIG. 4, the surface curvature may be convex on either or both of the front and back surfaces, similar to a conventional IOL. However, the curvature can be maintained relatively small to allow the IOL optics to be made thin while the diameter is increased to greater than 6.0 mm. Again, a significant portion of the total dioptric focusing power of the IOL (e.g., at least 20%, more preferably from 20 to 80% of the IOL dioptric power) may be derived from the internal gradient index of refraction layer in such relatively thin IOL. In a particular embodiment, most of the focusing power may be derived from the gradient index of refraction. The index gradient 23 within each concentric ring is such that the index is higher toward the center and lower peripherally. The concentric rings are analogous to a “Fresnel” lens, but with the difference that refraction is caused by internal index gradient (FIG. 5) rather than surface height gradient. Thus there is no surface ridges as in the traditional Fresnel lens. The widths of the rings may decrease with greater radius to provide a greater gradient peripherally to maintain uniform refractive power.

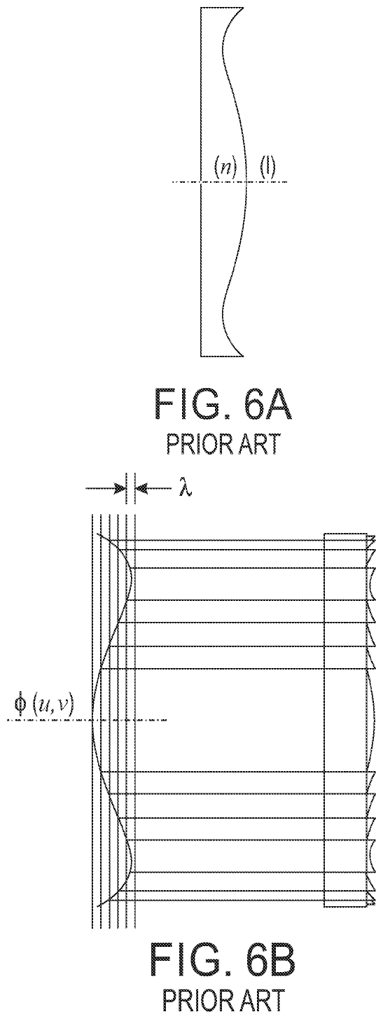

More particularly, use of concentric rings in such design enables further increase in total dioptric power in a relatively thin lens, as the design of the gradient index structures can be modified to reduce the range of refractive index variation required in the IOL optics to provide, e.g., a phase shift profile across the lens that is modulo-2π. Writing a phase shift profile in modulo-2π form is done by subtracting a constant phase shift of 2π from the total design phase shift in the regions where the total design phase shift is between 2π and 4π, subtracting a constant 4π phase shift from the total design phase shift in the regions where the total design phase shift is in the range 4π to 6π, etc., in a “Fresnel” lens type pattern so that only resulting net phase shifts of 0-2π need to be written in the lens. A Fresnel lens is much thinner and lighter than a continuous profile lens of the same diameter and focal length, since much of the bulk of the lens material is removed by Fresnel's design. The concept of the Fresnel lens is shown in FIG. 6b, for a corresponding conventional continuous refractive lens shown in FIG. 6a (reproduced from “The Phase Fresnel Lens,” K. Miyamoto, JOSA 51, 1, p. 17 (1959)). A continuous phase front is sampled into increments of 2π phase shift based on a design wavelength λ and a discontinuous phase structure is produced. In this type of Fresnel lens design, the sampled phase regions occupy the entire lens surface and the phase discontinuities do not exceed 2π anywhere.

One method of introducing a gradient index in an IOL of the present disclosure is irradiation with femtosecond laser pulses. U.S. Publication No. 2008/0001320, e.g., describes methods for modifying the refractive index of optical polymeric materials using very short pulses from a visible or near-IR laser having a pulse energy from 0.5 nJ to 1000 nJ, where the intensity of light is sufficient to change the refractive index of the material within the focal volume, whereas portions just outside the focal volume are minimally affected by the laser light. U.S. Publication No. 2009/0287306 describes a similar process to provide dioptric power changes in optical polymeric materials that contain a photosensitizer. The photosensitizer is present in the polymeric material to enhance the photoefficiency of the two-photon process used to form the refractive structures. In some instances, the rate at which the laser light is scanned across the polymeric material can be increased 100-fold with the inclusion of a photosensitizer and still provide a similar change in the refractive index of the material. U.S. Publication No. 2012/0310340 more particularly further describes a method for providing changes in refractive power of an optical device made of an optical, polymeric material by forming at least one laser-modified, gradient index (GRIN) layer disposed between an anterior surface and a posterior surface of the device by scanning with light pulses from a visible or near-IR laser along regions of the optical, polymeric material. The at least one laser-modified GRIN layer comprises a plurality of adjacent refractive segments, and is further characterized by a variation in index of refraction of at least one of: (i) a portion of the adjacent refractive segments transverse to the direction scanned; and (ii) a portion of refractive segments along the direction scanned. U.S. 2012/0310340 further discloses that the design of the gradient index structures can be modified to provide a phase shift that is modulo-2π to reduce the total device writing times. The disclosures of such patent publications are incorporated by reference herein.

The time required for such a writing procedure depends on how much total phase accumulation is required to create the desired refractive correction. Any medical procedure in humans will have time limits imposed by various factors, including safety, practicality, etc. In such a case, the use of Fresnel-type phase wrapped designs for the refractive corrector can be advantageous, since it enables writing the equivalent of a desired conventional continuous refractive phase shift pattern having a total maximum phase shift of greater than 2 in a refractive corrector with only a maximum net phase shift of 2π. Conventional continuous refracting lenses, however, have certain advantages over discontinuous Fresnel-type correctors. For instance, they have significantly lower chromatic aberration than Fresnel-type correctors, since they are simply limited by material dispersion. Furthermore they can in principle exhibit higher optical quality including lower scattering losses compared to Fresnel-type structures if the Fresnel-type structures do not have perfect phase discontinuities.

As the phase shifts between concentric rings are designed to be equal to the design wavelength or multiples of the design wavelength, the rings are not designed to form a diffractive pattern generating distinct foci at different distances, and thus is not designed to provide multi-focality. Accordingly, in particular embodiments, the intraocular lens is designed to be mono-focal for a design wavelength of the intraocular lens. In further embodiments, however, further design features may be incorporated into the lens if desired to additionally provide multi-focality.

In various embodiments, the internal optical feature in the intraocular lens provides a wavefront cross-section phase profile positively contributing to the total dioptric power of the intraocular lens, is established at least in part by laser machining the refractive corrector employing any of the laser-writing techniques referenced above.

In particular embodiments, the intraocular lens having an internal optical feature contributing to the total dioptric power may be formed by irradiating an optical, polymeric material with very short laser pulses of light as described in U.S. Publication Nos. 2008/0001320, 2009/0287306, 2012/0310340 and 2012/0310223 incorporated by reference above, where such short laser pulses are of sufficient energy such that the intensity of light within the focal volume will cause a nonlinear absorption of photons (typically multi-photon absorption) and lead to a change in the refractive index of the material within the focal volume, while the material just outside of the focal volume will be minimally affected by the laser light. The femtosecond laser pulse sequence pertaining to an illustrative embodiment, e.g., operates at a high repetition-rate, e.g., 80 MHz, and consequently the thermal diffusion time (>0.1 μs) is much longer than the time interval between adjacent laser pulses (˜11 ns). Under such conditions, absorbed laser energy can accumulate within the focal volume and increase the local temperature. This thermal mechanism likely plays a role in the formation of laser-induced refractive structures in optical, polymeric materials. Moreover, the presence of water in the polymeric material is believed to advantageously influence the formation of refractive structures. As such, optical hydrogel polymers provide much greater processing flexibility in the formation of the refractive structures as compared to zero or low water content optical polymers, e.g., the hydrophobic acrylates or low-water (1% to 5% water content) acrylate materials. The irradiated regions exhibit little or no scattering loss, which means that the resulting refractive structures that form in the focal volume are not clearly visible under appropriate magnification without phase contrast enhancement. In other words, the refractive structures are virtually transparent to the human eye without some form of image enhancement. An optical material is a polymeric material that permits the transmissions of at least 80% of visible light through the material, that is, an optical material does not appreciably scatter or block visible light.

According to one specific embodiment, an IOL is formed by providing an optical, polymeric lens material having an anterior surface and posterior surface and an optical axis intersecting the surfaces; and forming at least one laser-modified layer disposed between the anterior surface and the posterior surface with light pulses from a laser by scanning the light pulses along regions of the optical, polymeric material to cause changes in the refractive index of the polymeric lens material, so that the optical, polymeric lens material comprises an internal optical feature positively contributing to the total dioptric power of the intraocular lens.

Femtosecond laser pulse writing methods may be more advantageously carried out if an optical polymeric material, such as, e.g., an optical hydrogel material, includes a photosensitizer, as more particularly taught in U.S. Publication Nos. 2009/0287306 and 2012/0310340 incorporated by reference above. The presence of the photosensitizer permits one to set a scan rate to a value that is at least fifty times greater, or at least 100 times greater, than a scan rate without a photosensitizer present in the material, and yet provide similar refractive structures in terms of the observed change in refractive index of the material in the focal volume. Alternatively, the photosensitizer in the polymeric material permits one to set an average laser power to a value that is at least two times less, more particularly up to four times less, than an average laser power without a photosensitizer in the material, yet provide similar refractive structures. A photosensitizer having a chromophore with a relatively large multi-photon absorption cross section is believed to capture the light radiation (photons) with greater efficiency and then transfer that energy to the optical polymeric material within the focal volume. The transferred energy leads to the formation of the refractive structures and the observed change in the refractive index of the material in the focal volume.

A 60×0.70NA Olympus LUCPlanFLN long-working-distance microscope objective with variable spherical aberration compensation may be employed to laser-write refractive segments. As indicated by the following equation

$$Δ T ( r , z , t = 0 ) = \frac{β {{τ}_{P} [ I ( 0 , 0 ) ]}^{2} exp [ - 4 ( \frac{{r}^{2}}{{a}^{2}} + \frac{{z}^{2}}{{b}^{2}} ) ]}{{c}_{p} ρ}$$

the localized instantaneous temperature depends on both the pulse intensity and the magnitude of the two-photon absorption (TPA) coefficient. In order to produce an optical modification of a material that is of purely refractive character, i.e., non-absorbing or scattering, it is important to avoid optical damage, i.e., observed burning (scorching) or carbonization of the polymeric material. Such material or optical damage can be caused by excitation intensities exceeding a critical free-electron density. For hydrogel polymers containing a fair amount of water, the optical breakdown threshold is much lower than that in silica glasses. This breakdown threshold limits the pulse energy (in many cases to approximately 0.1 nJ to 20 nJ) that the hydrogel polymers can tolerate, and yet provide the observed changes in the refractive index within the focal volume.

Another way to increase energy absorption at a given intensity level is to increase the nonlinear absorption coefficient β by doping the optical, polymeric material with a particular chromophore and tuning the short pulse laser near a two-photon transition of the chromophore. In this regard, optical, hydrogel materials doped with a non-polymerizable photosensitizer or a polymerizable photosensitizer have been prepared. The photosensitizer will include a chromophore having a two-photon, absorption cross-section of at least 10 GM between a laser wavelength range of 750 nm to 1100 nm. In the former case of a non-polymerizable photosensitizer, solutions containing a photosensitizer may be prepared and the optical, hydrogel polymeric materials may be allowed to come in contact with such solutions to allow up-take of the photosensitizer into the polymeric matrix of the polymer. In the later case of a polymerizable photosensitizer, monomers containing a chromophore, e.g., a fluorescein-based monomer, may be used in the monomer mixture used to form the optical, polymeric material such that the chromophore becomes part of the polymeric matrix. Further, one could easily use a solution containing a non-polymerizable photosensitizer to dope an optical, polymeric material that had been prepared with a polymerizable photosensitizer. Also, it is to be understood that the chromophoric entities could be the same or different in each respective photosensitizer.

The concentration of a polymerizable, monomeric photosensitizer having a two-photon, chromophore in an optical material, preferably an optical, hydrogel material, can be as low as 0.05 wt. % and as high as 10 wt. %. Exemplary concentration ranges of polymerizable monomer having a two-photon, chromophore in an optical, hydrogel material is from 0.1 wt. % to 6 wt. %, 0.1 wt. % to 4 wt. %, and 0.2 wt. % to 3 wt. %. In various aspects, the concentration range of polymerizable monomer photosensitizer having a two-photon, chromophore in an optical, hydrogel material is from 0.4 wt. % to 2.5 wt. %.

Due to the high repetition rate pulse sequence used in the irradiation process, the accumulated focal temperature increase can be much larger than the temperature increase induced by a single laser pulse. The accumulated temperature increases until the absorbed power and the dissipated power are in dynamic balance. For hydrogel polymers, thermal-induced additional crosslinking within the polymer network can produce a change in the refractive index as the local temperature exceeds a transition temperature. If the temperature increase exceeds a second threshold, a somewhat higher temperature than the transition temperature, the polymer is pyrolytically degraded and carbonized residue and water bubbles are observed. In other words, the material exhibits visible optical damage (scorching). Each of the following experimental parameters such as laser repetition rate, laser wavelength and pulse energy, TPA coefficient, and water concentration of the materials should be considered so that a desired change in the refractive index can be induced in the hydrogel polymers without optical damage.

The pulse energy and the average power of the laser, and the rate at which the irradiated regions are scanned, will in-part depend on the type of polymeric material that is being irradiated, how much of a change in refractive index is desired and the type of refractive structures one wants to create within the material. The selected pulse energy will also depend upon the scan rate and the average power of the laser at which the refractive structures are written into the polymer material. Typically, greater pulse energies will be needed for greater scan rates and lower laser power. For example, some materials will call for a pulse energy from 0.05 nJ to 100 nJ or from 0.2 nJ to 10 nJ.

In one embodiment, the average pulse energy may be from 0.2 nJ to 10 nJ and the average laser power may be from 40 mW to 220 mW. The laser also operates within a wavelength of 650 nm to 950 nm. Within the stated laser operating powers, the optical, hydrogel polymeric material is irradiated at a scan rate, e.g., of from 0.4 mm/s to 4 mm/s. With higher laser powers, significantly faster scan speeds may be employed.

A photosensitizer will include a chromophore in which there is little or no intrinsic linear absorption in the spectral range of 600-1000 nm. The photosensitizer is present in the optical, hydrogel polymeric material to enhance the photoefficiency of the two-photon absorption required for the formation of the described refractive structures.

As described in U.S. Publication Nos. 2009/0287306 and 2012/0310340 in greater detail in the Example sections, a commercial IOL material, Akreos®, marketed by Bausch & Lomb, was subjected to laser irradiation according to the processes described therein. An Akreos® IOL is a HEMA-based, hydrogel material with 26% to 28% water content. The micromachining process was used to imprint refractive structures in an Akreos® IOL without photosensitizer and an Akreos® IOL doped with a solution containing 17 wt. % coumarin-1. The irradiation experiments were conducted with both dry and hydrated materials. The refractive structures formed only in the hydrated materials. In brief, the magnitude of the measured change in refractive index was at least ten times greater in the Akreos® IOL doped with the coumarin solution at a given scan rate and an average laser power than the Akreos® IOL without the coumarin.

In another illustrative aspect described in U.S. Publication Nos. 2009/0287306 and 2012/0310340, a balafilcon A silicone hydrogel was prepared by adding fluorescein monomer (0.17% by weight) as a polymerizable photosensitizer to the polymer monomer mixture. The balafilcon A doped with fluorescein was then subjected to laser radiation according to the processes described therein. Again, the described irradiation process was used to imprint refractive structures in the silicone hydrogel without photosensitizer and the silicone hydrogel doped with 0.17 wt. % fluorescein monomer. Again, experiments were conducted with both dry and hydrated materials, and again, the refractive structures formed only in the hydrated materials. In brief, the magnitude of the measured change in refractive index was at least ten times greater in the balafilcon A silicone hydrogel doped with 0.17 wt. % fluorescein monomer at an average laser power of 60 mW than balafilcon A without the photosensitizer. This 10-fold difference in change in refractive index was observed even with a 10-fold increase in scan rate in the photosensitized material; i.e., 0.5 mm/s in the undoped material and 5.0 mm/s in the photosensitized material.

The laser may generate light with a wavelength in the range from violet to near-infrared. In various aspects, the wavelength of the laser may be in the range from 400 nm to 1500 nm, from 400 nm to 1200 nm, or from 650 nm to 950 nm.

In an exemplary aspect, the laser may be a pumped Ti:sapphire laser with an average power of 10 mW to 1000 mW. Such a laser system will generate light with a wavelength of approximately 800 nm. In another exemplary aspect, an amplified fiber laser that can generate light with a wavelength from 1000 nm to 1600 nm may be used.

The laser may have a peak intensity at focus of greater than 10¹³ W/cm². At times, it may be advantageous to provide a laser with a peak intensity at focus of greater than 1014 W/cm², or greater than 1015 W/cm².

In addition to enabling relatively thin, large diameter intraocular lenses of desired total dioptric power to be inserted through relatively small incisions, the ability to form refractive structures in optical polymeric materials provides an important opportunity to an ophthalmic surgeon or practitioner to modify the refractive index of an intraocular lens following implantation of the device into an eye of a patient. The method in particular further allows the surgeon to correct aberrations as a result of the surgery. The method also allows the surgeon to adjust the refractive properties of the lens by adjusting the refractive index in the irradiated regions based on the vision correction required of each patient. For example, starting from a lens of selected power (will vary according to the ocular requirements of the patient), the surgeon can further adjust the refractive properties of the lens to correct a patient's vision based upon the individual needs of the patient. In essence, an intraocular lens would essentially function like a contact lens or glasses to individually correct for the refractive error of a patient's eye. Moreover, because the implanted lens can be adjusted by adjusting the refractive index of select regions of the lens, post-operative refractive errors resulting from pre-operative measurement errors, variable lens positioning during implantation, and wound healing (aberrations) can be corrected or fine tuned in-situ.

Typically, the irradiated portions of the optical polymeric lens material will exhibit a change in refractive index of at least about 0.01 or more. In one embodiment, the refractive index of the region will change by about 0.03 or more. As disclosed in U.S. Publication Nos. 2009/0287306 and 2012/0310340, a change in refractive index in a hydrated, Akreos® IOL material of about 0.06 has been measured.

In an exemplary aspect, the irradiated regions of an optical, polymeric material can be defined by two- or three-dimensional structures providing the desired wavefront cross-section profile. The two- or three-dimensional structures can comprise an array of discrete cylinders, a series of lines, or a combination of an array of cylinders and a series of lines. Moreover, the two- or three-dimensional structures can comprise area or volume filled structures, respectively. These area or volume filled structures can be formed by continuously scanning the laser at a constant or varying scan rate over a selected region of the polymeric material. Nanometer-sized structures can also be formed by the zone-plate-array lithography method describe by R. Menon et al., Proc. SPIE, Vol. 5751, 330-339 (May 2005); Materials Today, p. 26 (February 2005).

In one aspect, the refractive structures may be formed proximate to the top anterior surface of an intraocular lens. For example, a positive or negative lens element (three-dimensional) is formed within a 300 μm volume, or within a 100 μm volume, from the anterior surface of the lens. The term “anterior surface” is the surface of the lens that faces the anterior chamber of a human eye.

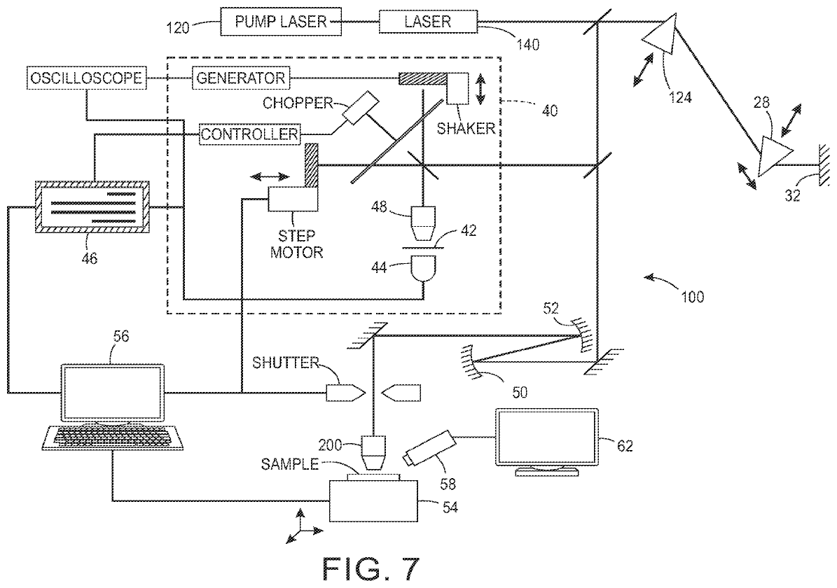

A non-limiting embodiment of a laser system 100 which may be used for irradiating an optical, polymeric material with a laser to modify the refractive index of the material in select regions to form an internal optical feature providing a wavefront cross-section phase profile as described herein is illustrated in FIG. 7. A laser source comprises a Kerr-lens mode-locked Ti:Sapphire laser 120 (Kapteyn-Murnane Labs, Boulder, Colorado) pumped by 4 W of a frequency-doubled Nd:YVO4 laser 140. The laser generates pulses of 300 mW average power, 30 fs pulse width, and 93 MHz repetition rate at wavelength of 800 nm. Because there is a reflective power loss from the mirrors and prisms in the optical path, and in particular from the power loss of the objective 200, the measured average laser power at the objective focus on the material is about 120 mW, which indicates the pulse energy for the femtosecond laser is about 1.3 nJ.

Due to the limited laser pulse energy at the objective focus, the pulse width must be preserved so that the pulse peak power is strong enough to exceed the nonlinear absorption threshold of the materials. Because a large amount of glass inside the focusing objective significantly increases the pulse width due to the positive dispersion inside of the glass, an extra-cavity compensation scheme is used to provide the negative dispersion that compensates for the positive dispersion introduced by the focusing objective. Two SF10 prisms 24 and 28 and one ending mirror 32 form a two-pass, one-prism-pair configuration. In a particular instance, a 37.5 cm separation distance between the prisms is used to compensate for the positive dispersion of the microscope objective and other optics within the optical path.

A collinear autocorrelator 40 using third-order harmonic generation is used to measure the pulse width at the objective focus. Both 2^{nd} and 3^{rd} harmonic generation have been used in autocorrelation measurements for low NA or high NA objectives. Third-order surface harmonic generation (THG) autocorrelation may be used to characterize the pulse width at the focus of the high-numerical aperture (NA) objectives because of its simplicity, high signal to noise ratio, and lack of material dispersion that second harmonic generation (SHG) crystals usually introduce. The THG signal is generated at the interface of air and an ordinary cover slip 42 (Corning No. 0211 Zinc Titania glass), and measured with a photomultiplier 44 and a lock-in amplifier 46. After using a set of different high-numerical-aperture objectives and carefully adjusting the separation distance between the two prisms and the amount of glass inserted, a transform-limited 27 fs duration pulse is used, which is focused by a 60×0.70NA Olympus LUCPlanFLN long-working-distance objective 48.

Because the laser beam will spatially diverge after it comes out of the laser, a concave mirror pair 50 and 52 is added into the optical path in order to adjust the dimension of the laser beam so that the laser beam can optimally fill the objective aperture. A 3D 100 nm resolution DC servo motor stage 54 (Newport VP-25XA linear stage) and a 2D 0.7 nm resolution piezo nanopositioning stage (PI P-622.2CD piezo stage) are controlled and programmed by a computer 56 as a scanning platform to support and locate the samples. The servo stages have a DC servo-motor so they can move smoothly between adjacent steps. An optical shutter controlled by the computer with 1 ms time resolution is installed in the system to precisely control the laser exposure time. With customized computer programs, the optical shutter could be operated with the scanning stages to micromachine different patterns in the materials using different scanning speeds at different position or depth in the optical material, and different laser exposure times. In addition, a CCD camera 58 along with a monitor 62 is used beside the objective 20 to monitor the process in real time.

The method and optical apparatus described above can be used to modify the refractive index of an intraocular lens following the surgical implantation of the intraocular lens in a human eye, or before the lens is implanted in an eye.

Accordingly, an embodiment described herein is directed to a method comprising identifying and measuring the aberrations resulting from the surgical procedure of providing a patient with an IOL. Once the aberrations are identified and quantified using methods well known in the art of ophthalmology, this information is processed by a computer. Of course, information related to the requisite vision correction for each patient can also be identified and determined, and this information can also be processed by a computer. There are a number of commercially available diagnostic systems that are used to measure the aberrations. For example, common wavefront sensors used today are based on the Schemers disk, the Shack Hartmann wavefront sensor, the Hartmann screen, and the Fizeau, and Twyman-Green interferometers. The Shack-Hartmann wavefront measurement system is known in the art and is described in-part by U.S. Pat. Nos. 5,849,006; 6,261,220; 6,271,914 and 6,270,221. Such systems operate by illuminating a retina of the eye and measuring the reflected wavefront.

Once the aberrations are identified and quantified, the computer programs determine the position and shape of the refractive structures to be written into the lens material to correct for those aberrations or to provide vision correction to the patient. These computer programs are well known to those of ordinary skill in the art. The computer then communicates with the laser-optical system and select regions of the lens are irradiated with a laser having a pulse energy from 0.05 nJ to 1000 nJ as described herein, to provide a desired modified wavefront cross-section phase profile. In accordance with the present disclosure, such modified wavefront cross-section phase profile is designed at least in part to provide added dioptric power in a relatively large diameter IOL so as to enable large diameter IOLs with a relatively higher dioptric power while maintaining lens cross-sectional area along the lens diameter to dimensions suitable for insertion through a relatively small limbal incision.

The optical, polymeric materials that can be irradiated with a laser according to the methods described to form refractive correctors in accordance with various embodiments can be any optical, polymeric material known to those of ordinary skill in the polymeric lens art, particularly those in the art familiar with optical polymeric materials used to make intraocular lenses. Non-limiting examples of such materials include those used in the manufacture of ophthalmic devices, such as siloxy-containing polymers, acrylic, hydrophilic or hydrophobic polymers or copolymers thereof. The forming of the refractive structures is particularly suited for modifying the refractive index in select and distinct regions of a polymeric, optical silicone hydrogel, or a polymeric, optical non-silicone hydrogel.

The term “hydrogel” refers to an optical, polymeric material that can absorb greater than 10% by weight water based on the total hydrated weight. In fact, many of the optical, hydrogel polymeric materials will have a water content greater than 15% or greater than 20%. For example, many of the optical, hydrogel polymeric materials will have a water content from 15% to 60% or from 15% to 40%.

The optical, polymeric materials are of sufficient optical clarity, and will have a relatively high refractive index of approximately 1.40 or greater, particularly 1.48 or greater. Many of these materials are also characterized by a relatively high elongation of approximately 80 percent or greater.

Without exclusion as to any lens materials or material modifications, e.g., the inclusion of a photosensitizer, or laser parameters described herein above, the foregoing disclosed techniques and apparatus can be used to modify the refractive properties, and thus, the dioptric power, of an optical polymeric material, typically, an optical hydrogel material, in the form of, but not limited to, an IOL or corneal inlay, by creating (or machining) a refractive structure with a gradient index in one, two or three dimensions of the optical material, as more fully described in U.S. Publication Nos. 2012/0310340 and 2012/0310223, incorporated by reference above. The gradient refractive structure can be formed by continuously scanning a continuous stream of femtosecond laser pulses having a controlled focal volume in and along at least one continuous segment (scan line) in the optical material while varying the scan speed and/or the average laser power, which creates a gradient refractive index in the polymer along the segment. Accordingly, rather than creating discrete, individual, or even grouped or clustered, adjoining segments of refractive structures with a constant change in the index of refraction in the material, a gradient refractive index is created within the refractive structure, and thereby in the optical material, by continuously scanning a continuous stream of pulses. As described in greater detail in U.S. Publication No. 2012/0310340, since the refractive modification in the material arises from a multiphoton absorption process, a well-controlled focal volume corrected for spherical (and other) aberrations will produce a segment having consistent and, if desired, constant depth over the length of the scan. As further noted, when a tightly focused laser beam consisting of femtosecond pulses at high repetition rate impinges on a material that is nominally transparent at the incident laser wavelength, there is little if any effect on the material away from the focal region. In the focal region, however, the intensity can exceed one terawatt per square centimeter, and the possibility of absorbing two or more photons simultaneously can become significant. In particular, the amount of two-photon absorption can be adjusted by doping or otherwise including in the irradiated material with selected chromophores that exhibit large two-photon absorption cross-section at the proper wavelength (e.g., between 750 nm and 1100 nm), which can significantly increase the scanning speed as already described. Also, multiple segments can be written into the material in a layer using different scan speeds and/or different average laser power levels for various segments to create a gradient index profile across the layer, i.e., transverse to the scan direction. Further, multiple, spaced gradient index (GRIN) layers can be written into the material along the z-direction (i.e., generally the light propagation direction through the material) to provide a desired refractive change in the material that provides a significant added dioptric power or that otherwise corrects for some, most, or all higher order aberrations of a patient's eye. Such abilities to write continuously varying gradient index layers are particularly advantageous in forming refractive correctors having wavefront cross-section profiles in accordance with embodiments of the present invention. For ophthalmic applications, it is of particular interest that GRIN refractive structures are low scattering (as discussed above) and are of high optical quality.

In an illustrative aspect disclosed in U.S. Publication No. 2012/0310340, a cylindrical lens structure with a one-dimensional quadratic gradient index was written in an optical, polymeric material with three GRIN layers each 5 μm thick, spaced by 10 μm in the z-direction (i.e., a layer of non-modified optical material having a thickness of about 5 μm to 7 μm was between each two adjacent GRIN layers). The resulting cylindrical lens was designed to provide approximately 1 diopter of astigmatism uniform along the length of the device.

### Example 1

An Intraocular lens as illustrated in FIGS. 1 and 2 may be obtained by the femtosecond laser writing process as described herein writing internal optical features in the form of a gradient index layer extending essentially across the entire optics region so as to positively contribute to the total dioptric power of the IOL.

In FIG. 2, the effects of the laser pulses are represented by dots 13 which is more in the center than in the periphery. Such a dot arrangement would be employed where the change in refractive index is positive, so that the center of the lens has a higher refractive index then the periphery, so as to provide a positive added optical power. Where the change in refractive index is negative, however, the reverse arrangement may be provided, i.e., more dots at the periphery, so as to create an internal gradient lens structure still providing a positive added optical power.

While FIG. 2 depicts spots representing refractive index change in a relatively thick three-dimensional volume extending through the thickness of the IOL, as described in W. Knox “Inventing a New Way to See Clearly: Non-Invasive Vision Correction With Femtosecond Lasers,” Technology and Innovation, Vol. 20, pp. 385-398 (2019), individual GRIN layers written in optical lens materials by such femtosecond laser writing processes by varying the laser exposure during a scan (e.g., by varying the scan rate and/or laser power during a scan) may measure in the range of 3 to 5 microns and as a result the amount of phase shift in a single layer may be limited depending upon the specific material employed (e.g., to 0.1 or 0.2 waves of phase shift in a single layer for some materials, or up to 4 to 6 waves of phase shift in a single layer for other materials). Accordingly, in such embodiment, an appropriate number of overlapping GRIN layers may be written to provide a desired added dioptric power, depending upon the specific lens material employed and the obtainable phase shift per written GRIN layer. The total possible added dioptric power may be limited by practical numbers of overlapping written layers (e.g., less than or equal to about 10 layers in preferred embodiments).

While the illustrated embodiment of FIGS. 1 and 2 has been described primarily with regard to formation by femtosecond laser writing, other methods known to those skilled in the art to produce index gradient in polymer lenses may be employed, such as diffusion of diluent or monomers, such as disclosed in Moore U.S. Pat. No. 3,718,383, the disclosure of which is incorporated by reference herein. One method of controlling monomer diffusion is the non-uniform application of ultraviolet (UV) light. Platt et al. U.S. Pat. No. 7,105,110, the disclosure of which is incorporated by reference herein, e.g., discloses the use of UV light to make small adjustments in IOL power after surgery, but does not teach to produce changes to total dioptric power so as to enable relatively large diameter, thin IOLs of sufficient high desired total dioptric power to be insertable through relatively small incisions.

### Example 2

An Intraocular lens as illustrated in FIGS. 3 and 4 may be obtained by the femtosecond laser writing process as described herein writing internal optical features in the form of concentric rings in a layer wherein each ring is itself a gradient index layer so as to form a gradient index Fresnel type lens structure which positively contributes to the total dioptric power of the IOL. In such embodiments, as each ring may utilize the maximum phase shift obtainable in a single layer, greater phase shift may be collectively enabled by each Fresnel type GRIN layer across the optics area, resulting in the ability to prepare IOLs with greater added dioptric powers, or to prepare IOLs with fewer written GRIN layers thereby improving manufacturability. When each ring (or zone) of the Fresnel GRIN layer achieves a full wavelength of phase change, the number of zones in a modulo-2π phase-wrapped Fresnel structure depends on the radius (R) of the optics, wavelength (λ), and dioptric power (D) in accordance with the following formula:

Number of Zones=ceiling[R2*D/(2λ)]
wherein the ceiling function is used to round up to an integer number. Thus, the GRIN layer may be designed to provide a desired added dioptric power for a design wavelength by specifying the number of zones in the GRIN layer for an IOL of specified diameter. If the number of zones is practically too large for manufacturability in a single layer, the desired dioptric power may be obtained by writing multiple stacked GRIN layers.

In specific examples of embodiments in accordance with FIGS. 1-5, IOLs may be designed from commercially available IOL materials to provide equivalent dioptric powers as in commercially available lens made with the same IOL materials in a relatively larger diameter, relatively thinner lens having lower or nearly equivalent cross-sectional area so as to maintain insertability through small incisions. In further embodiments, relatively thinner lens having internal optical features providing added optical power may further enable insertion of a lens having a desired optic portion diameter and a desired total dioptric power through even small incisions that might not otherwise be achievable without such internal optical features, such as an incision of 2.0 mm or less, or even of 1.5 mm or less.

Table II below, e.g., describe physical dimensions and properties for representative relatively high power IOLs, with optics diameters of 6 mm and comprising commercially available optical materials having refractive indexes of 1.47 and 1.55, respectively designated IOL A and IOL B (employing acrylic lens materials as similarly employed in TECNIS™ and ACRYSOF™ IOLs, respectively). IOLs A′ and B′, on the other hand, describe physical dimensions and properties for larger diameter (7 mm and 8 mm, respectively), thinner IOLs of the same materials as IOLs A and B, having the same cross-sectional areas as each of A and B. As indicated, in order to maintain equivalent cross-sectional areas as the smaller IOLs A and B, such larger, thinner IOLs A′ and B′ provide lower dioptric powers based solely on the curvature of the lens surfaces compared to the smaller, thicker IOLs A and B having relatively greater curvature. In accordance with an embodiment of the present disclosure, additional desired dioptric power may be added by forming one or more GRIN layer internally in the IOLs, thus maintaining a desired cross-sectional area in a relatively high powered, larger diameter IOL so as to be insertable through an injector for a small incision. In specific embodiments, e.g., the internal optical feature is designed to provide from about +1 D to about +20 D added dioptric power.

In further embodiments, additional IOLs A″ and B″ are provided. As indicated in Table II, in order to obtain an even smaller cross-sectional areas than the smaller IOL A and the larger IOL A′, such larger IOL A″ is made even thinner by further reducing the lens surface curvature, which results in IOL A″ providing only about half of the dioptric power as the smaller, thicker IOL A. For IOL B″, on the other hand, the lens edge thickness is reduced relative to that of IOLs B and B′, enabling an increase in surface curvature and resulting dioptric power relative to IOL B′, while maintaining nearly equivalent cross-sectional areas as IOLs B and B″, again providing about half of the dioptric power as the smaller, thicker IOL B. In accordance with an embodiment of the present disclosure, the other half of the desired dioptric power (or a lower or higher fraction if desired) again may be added by forming a GRIN layers internally in the IOLs, thus maintaining a desired cross-sectional area in a relatively high powered IOL so as to be insertable through an injector for a small incision.

TABLE II

| | | | | | Sagittal | cross |
|---|---|---|---|---|---|---|
| | Diam. | power | | edge | thickness | section |
| IOL | (mm) | (D) | index | (mm) | (mm) | (mm²) |
| A. | 6 | 34 | 1.47 | 0.02 | 0.3455 | 1.422 |
| A′. | 7 | 21.1 | 1.47 | 0.02 | 0.2948 | 1.422 |
| A″. | 7 | 17 | 1.47 | 0.02 | 0.2415 | 1.174 |
| B. | 6 | 30 | 1.55 | 0.03 | 0.2755 | 1.282 |
| B′. | 8 | 13.4 | 1.55 | 0.03 | 0.2253 | 1.282 |
| B″. | 8 | 15 | 1.55 | 0.02 | 0.2382 | 1.324 |

Of the manufacturing processes that could be used to form index gradient in intraocular lenses comprising polymeric optical materials, femtosecond laser pulses or UV irradiation are most suitable for producing the complex concentric pattern of this embodiment.

### Example 3

While FIGS. 1-2 illustrate a single continuous gradient index feature extending across the optic portion 12 and FIGS. 3-5 illustrate a discontinuous modulo-2π embodiment, in a further example a relatively thin, large diameter IOL instead may employ a combination of such features, similarly as taught in US 2018/0373060, the disclosure of which is incorporated by reference herein. In accordance with such embodiment, the intraocular lens may be provided with an internal optical feature comprising: (a) a central zone having a continuous wavefront cross-section phase profile, having a wavefront maximum height of greater than 1 design wavelength λ in the area of the central zone; and (b) a peripheral region comprising multiple segments and having a discontinuous wavefront cross-section phase profile having phase shifts between segments that are equal to the design wavelength or multiples of the design wavelength, and wherein the phase shifts in the peripheral region are less than or equal to the wavefront maximum height in the central zone.

In addition to providing a positive contribution to the total dioptric power, the described internal optical features can be employed for additionally providing other Wavefront phase profiles, such as, e.g., hyperbolic phase profiles, aspheric phase profiles, toric, and free-form/arbitrary phase profiles. Hyperbolic phase profiles may be employed, e.g., for adding multifocality, as well as other known uses. Aspheric phase profiles with spherical aberrations (4th order and higher), may be employed to extend the depth of focus. Free-form/arbitrary phase profiles may be employed, e.g., to correct the native higher order aberration profiles of individuals.

It will be appreciated that variants of the above-disclosed embodiments and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. A foldable intraocular lens (IOL) having a total dioptric power and comprising an optic portion having main anterior and posterior optical surfaces and an optical axis intersecting the surfaces, and incorporating an internal optical feature positioned between the main anterior and posterior optical surfaces, wherein the optic portion has a diameter of greater than 6.0 mm and a maximum central cross-sectional area of less than 2 mm² along the diameter, and wherein the internal optical feature positively contributes to the total dioptric power of the intraocular lens.

2. An intraocular lens according to claim 1, having a total dioptric power of from about 5 D to about 40 D in aqueous and vitreous humors of the human eye.

3. An intraocular lens according to claim 1, wherein the internal optical feature provides at least 20% of the total dioptric power of the IOL.

4. An intraocular lens according to claim 3, wherein the internal optical feature provides from 20% to 80% of the total dioptric power of the IOL.

5. An intraocular lens according to claim 1, wherein the main anterior and posterior optical surfaces are convex surfaces providing a refractive optical power component of the total dioptric power of the intraocular lens of +5 to +20 D in aqueous and vitreous humors of the human eye.

6. An intraocular lens according to claim 5, wherein the internal optical feature provides from about +1 to about +20 added dioptric power, such that the IOL provides a desired dioptric power of from about +6 to about +40 dioptric power.

7. An intraocular lens according to claim 5, wherein the internal optical feature provides from about +3 to about +20 added dioptric power, such that the IOL provides a desired dioptric power of from about +8 to about +40 dioptric power.

8. An intraocular lens according to claim 5, wherein the internal optical feature provides from about +5 to about +20 added dioptric power, such that the IOL provides a desired dioptric power of from about +10 to about +40 dioptric power.

9. An intraocular lens according to claim 1, wherein the optic portion has a diameter of 6.1-10 mm.

10. An intraocular lens according to claim 1, wherein the optic portion has a diameter of 6.3-9 mm.

11. An intraocular lens according to claim 1, wherein the optic portion has a diameter of 6.5-8 mm.

12. An intraocular lens according to claim 1, wherein the optic portion has a diameter of 6.5-7.5 mm.

13. An intraocular lens according to claim 1, wherein the optic portion has a diameter of 6.5-7.0 mm.

14. The intraocular lens of claim 1, wherein the optic portion has a maximum central cross-sectional area of less than or equal to 1.53 mm² along the diameter.

15. The intraocular lens of claim 1, wherein the optic portion has a maximum central cross-sectional area of less than or equal to 1.3 mm² along the diameter.

16. The intraocular lens of claim 1, wherein the optic portion has a maximum central cross-sectional area of less than or equal to 1.0 mm² along the diameter.

17. The intraocular lens of claim 1, wherein the internal optical feature comprises one or more gradient index layer extending across the optic portion.

18. The intraocular lens of claim 1, wherein the internal optical feature comprises one or more gradient index layer providing a phase profile sampled into 2π phase discontinuities.

19. The intraocular lens of claim 1, wherein the intraocular lens is mono-focal for a design wavelength.

20. The intraocular lens of claim 19, wherein the design wavelength is a wavelength between 400 and 700 nm.

21. The intraocular lens of claim 19, wherein the design wavelength is 555 nm.

22. A method of forming an intraocular lens having a total dioptric power in accordance with any of claims 1-21, the method comprising:
providing a foldable intraocular lens having an optic portion comprising an optical, polymeric lens material and having an anterior surface and posterior surface and an optical axis intersecting the surfaces; and
forming at least one laser-modified layer disposed between the anterior surface and the posterior surface with light pulses from a laser by scanning the light pulses along regions of the optical, polymeric material to cause changes in the refractive index of the polymeric lens material;
wherein the optic portion has a diameter of greater than 6.0 mm and a maximum central cross-sectional area of less than 2.0 mm² along the diameter, and wherein the laser-modified layer forms an internal optical feature that positively contributes to the total dioptric power of the intraocular lens.

23. The method of claim 22, wherein the at least one laser-modified layer includes a portion having a continuous variation in index of refraction.

24. The method of claim 22, wherein the at least one laser-modified layer includes portions having discontinuous variations in index of refraction forming region.

25. A method of inserting an intraocular lens according to any of claims 1-21 into an eye, the method comprising: making an incision of less than 3.0 mm length, and inserting a folded intraocular lens through the incision, wherein the intraocular lens has a total dioptric power and comprises an optic portion having main anterior and posterior optical surfaces, a diameter of greater than 6.0 mm and a maximum central cross-sectional area of less than 2 mm² along the diameter, and an internal optical feature positioned between the main anterior and posterior optical surfaces, wherein the internal optical feature positively contributes to the total dioptric power of the intraocular lens.

26. The method of claim 25, wherein the incision is made with a length of 2.6 mm or less, and the intraocular lens has maximum cross-sectional area of about 1.53 mm² or less along the diameter.

27. The method of claim 25, wherein the incision is made with a length of 2.2 mm or less, and the intraocular lens has maximum cross-sectional area of about 1.1 mm² or less along the diameter.

28. The method of claim 25, wherein the incision is made with a length of 2.0 mm or less, and the intraocular lens has maximum cross-sectional area of about 0.91 mm² or less along the diameter.
